(19) United States
(12) Patent Application Publication
(10) Pub. No.: US 2006/0241395 A1
(43) Pub. Date: Oct. 26, 2006

(54) Device and method for locating an instrument within a body

(76) Inventors: Sascha Kruger, Hamburg (DE); Jorn Borgert, Hamburg (DE)

Correspondence Address: PHILIPS INTELLECTUAL PROPERTY & STANDARDS, 595 MINER ROAD, CLEVELAND, OH, 44143, US

(21) Appl. No.: 10/548,334

(22) PCT Filed: Mar. 3, 2004
(86) PCT No.: PCT/IB04/00556

§ 371 Date: Sep. 7, 2005

(30) Foreign Application Priority Data
Mar. 7, 2003 (EP) 03100567.1

(51) Int. Cl. A61B 5/05

(52) U.S. Cl. 600/424

(57) ABSTRACT

The invention relates to a device and a method for locating an instrument, such as a catheter (104) for example, within a body (106). The catheter (104) has a number of light guides into which there is passed an NIR radiation pulse (102) from a laser (101). The NIR radiation is emitted by scattering end sections (105) of the light guides into the body volume (106) and detected outside the body by means of cameras (107a, 107b, 107c). Scattered photons are preferably excluded by means of a temporally selective amplification. The location of the catheter (104) can be reconstructed stereoscopically on the basis of the camera images.

The invention relates to a device and a method for locating an instrument, such as a catheter in particular, within a body, and also to a catheter that is suitable for this purpose.

U.S. Pat. No. 6,264,610 B1 discloses a probe which from a body region that is to be examined generates images simultaneously by means of ultrasound and by means of light of the near infrared (NIR). In this way, it is possible for the advantages of good spatial resolution of internal structures on account of the ultrasound and the detection of chemical compositions such as the oxygen content for example on account of the NIR light to be combined. By combining two different techniques, however, the device is very complex. Furthermore, it does not include any means for locating an object within a body.

The extremely precise location of an instrument that has been inserted into a body and is thus no longer visible, such as a catheter in the vascular system of a patient for example, is generally highly important in respect of the diagnostic or therapeutic use of the instrument. The most significant known locating techniques in this connection are based either on ultrasound or on magnetism. Ultrasound systems use the propagation time of an ultrasound signal through the body for the purpose of distance determination. However, since the sound velocity is very different in different body tissues and there are usually a number of different tissue types between the ultrasound source and the receiver, ultrasound systems are relatively inaccurate in medical applications and are therefore limited in terms of the extent to which they can be used. Magnetic systems encounter difficulties when there are iron-containing or electrically conductive materials in the vicinity of the locating system. However, since this is the case in many medical applications, the usability and reliability of these systems in medicine is also limited.

Against this background, it is an object of the present invention to provide means for the reliable locating of an instrument, such as a catheter in particular, within a body.

This object is achieved by a method having the features of claim 1, a device having the features of claim 7 and a catheter having the features of claim 10. Advantageous refinements are given in the dependent claims.

The method according to the invention is used to locate an instrument within a body. The instrument may be in particular a catheter which is surrounded for example by biological tissue. The method comprises the following steps:

a) The emission of radiation from the near infrared (NIR) range, that is to say having a wavelength of typically 0.65 μm to 3 μm, coming from at least one emission point on the instrument.

b) The detection of the NIR radiation, emitted according to step a), outside the body.

c) The reconstruction of the spatial position of the emission point on the basis of the NIR radiation detected outside the body in step b).

The method described makes use of the fact that NIR radiation is absorbed by many substances to a lesser extent than visible light. In particular, a considerable fraction of NIR radiation may pass through layers of biological tissue having a typical thickness of a few tens of centimeters, so that it can be detected outside the tissue. A further advantage of NIR radiation is that it is to a large extent unharmful to biological tissue. The intensity and duration of irradiation can therefore where appropriate be adapted such that desired imaging properties are achieved.

There are various possibilities for reconstructing the spatial position of a point of emission of NIR radiation based on the radiation detected outside a body. Preferably, the detection of the NIR radiation emitted in step a) of the method takes place in parallel at a number of locations outside the body, with the position of the emission point being stereoscopically reconstructed from the information obtained. In such a stereoscopic reconstruction, the direction from which the NIR radiation comes from the emission point, as seen from the respective location, is determined at at least two different locations. The point of intersection of these directions then corresponds to the position of the emission point. If the emission point lies on the connecting line between two observation locations, its position cannot be determined unambiguously. In order to confront such cases and increase the accuracy of the method in general by means of redundant measurements, the radiation detection preferably takes place at at least three different locations outside the body.

In many cases, it is desirable to know the position of a number of points on an instrument. By way of example, in the case of a catheter the spatial orientation of the catheter tip and/or the spatial form of a deformable catheter section may be of great interest. In these cases, the method described is preferably carried out for a number of points of emission of NIR radiation located at various sites on the instrument. The NIR radiation is advantageously emitted from the various emission points at different points in time, that is to say sequentially, so that at each observation time it can be unambiguously ascertained from which emission point detected radiation must have come.

According to a preferred embodiment of the method, the NIR radiation is emitted as a short time pulse. The duration of such a pulse is typically 0.1 to 10 ps, preferably around 1 ps. Such pulses of NIR radiation may be generated by conventional lasers and prove to be sufficient for the necessary detection. One significant advantage of short pulses is that the width thereof lies in or below the order of magnitude of the time loss experienced by the photons on account of scattering on their route through the body. Scattered photons therefore lie significantly outwith the original pulse form or pulse duration.

In one preferred embodiment of the method, only photons of direct radiation, which take the direct route from the emission point to the detection location without undergoing any scattering processes, are used for the detection of the NIR radiation outside the body. Limiting the detection to photons of direct radiation considerably increases the accuracy of the position determination since scattered photons generally do not come from the direction of the emission point and therefore falsify any conclusions drawn about the position thereof. Since in biological tissue a great number of scattering processes, sometimes also multiple scattering processes, of the photons generally take place, exclusion thereof from the detection process is highly important for medical applications. The exclusion of scattered photons may in particular be based on the taking into account of the propagation time of the photons. From the time window, only photons corresponding to direct radiation are used for the detection. Scattered photons require a longer propagation time and therefore no longer reach the detection point within this time window.

According to one preferred embodiment of the method, the above-described limitation of the detection to photons of direct radiation is achieved in that the photons of the emitted NIR radiation are irradiated into an activated amplification medium, where they are amplified by induced emissions. In order to terminate this amplification, a quench pulse which deactivates the amplification medium is irradiated into the amplification medium at a desired point in time. In this way, only the early photons of (direct) NIR radiation which arrive before the quench pulse are amplified, while the (scattered) photons which arrive later remain unamplified.

Further details regarding the abovementioned method are to be taken from the patent application having the title “Device and method for the selective amplification of photons in a time window”, filed by the same applicant at the same time, the contents of which are hereby incorporated by way of reference into the present application.

The invention furthermore relates to a device for locating an instrument, such as a catheter in particular, within a body, which device comprises the following components:

a) at least one detector for the locally resolved detection of NIR radiation outside the body, said NIR radiation coming from at least one emission point of the instrument;

b) means for reconstructing the position of the emission point from the measured values of the detector.

Said device can be used to carry out the abovementioned method so that the advantages thereof can be obtained. The device can be further developed such that it can also be used to carry out the described variants of the method.

In particular, the detector of the device may have a time window filter unit for the selective detection of photons from a predefined time window. The time window is preferably set such that it contains the photons of direct radiation which pass from the emission point to the detector without undergoing any scattering processes and screens out scattered photons of an NIR radiation pulse.

The time window filter unit may be formed by an activatable amplification medium (e.g. a laser medium) and a quenching device for irradiating a quench pulse into the amplification medium. In the activated state of the amplification medium, NIR radiation that is passed into the latter is amplified by induced emissions. This amplification may be terminated at a desired point in time by the emitting of a quench pulse by the quenching device, so that the amplification remains limited to a desired time window.

The invention furthermore relates to a catheter for use in a method of the type mentioned above, said catheter comprising a number of NIR light guides. The light guides each have a highly NIR light-scattering section that acts as an emission point for emitting NIR radiation into the body during use of the catheter. The light guides furthermore each have an inlet for the coupling-in of NIR pulses. When such a catheter is inserted into the body, NIR pulses can be transmitted via the inlets along the light guides, said NIR pulses being emitted into the interior of the body at the scattering sections. The position of the scattering sections can then be located in a method or using a device of the abovementioned type. The described design of the catheter is preferably combined with other catheter functions of diagnostic or therapeutic nature.

The invention will be further described with reference to examples of embodiments shown in the drawings to which, however, the invention is not restricted. Identical components are provided in the figures with identical references and are therefore in general only described once.

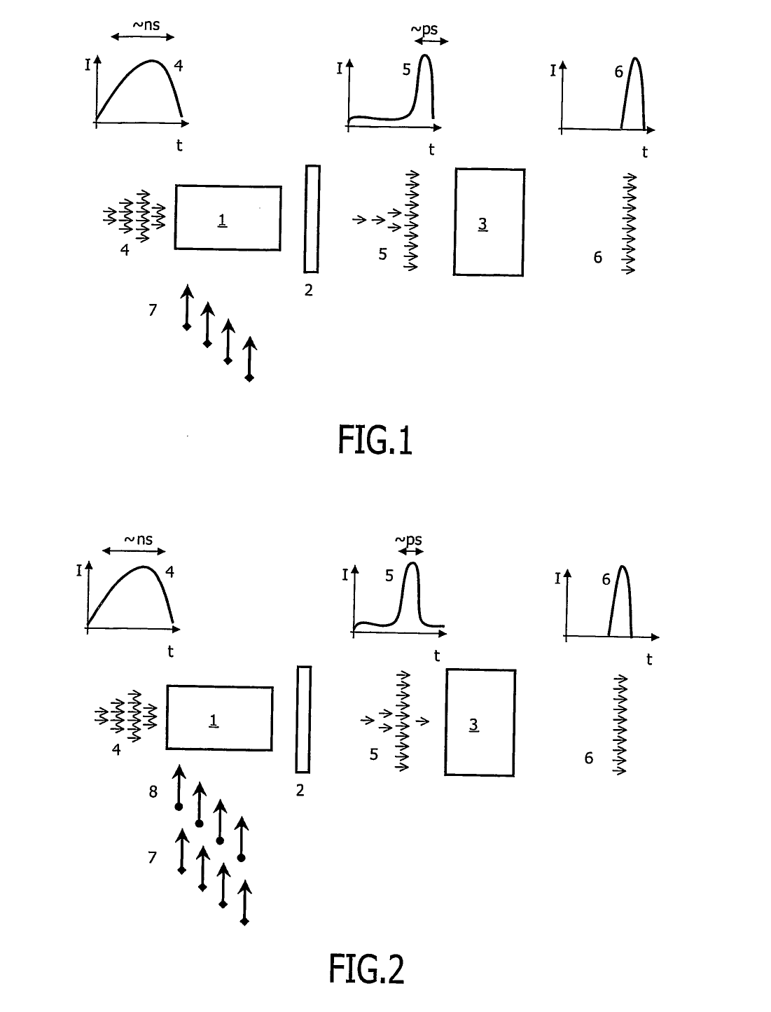

FIG. 1 shows the principle of amplification of a signal photon pulse up to irradiation of a quench pulse.

FIG. 2 shows a variant of the method of FIG. 1, in which the start of amplification is defined by the irradiation of a pump pulse.

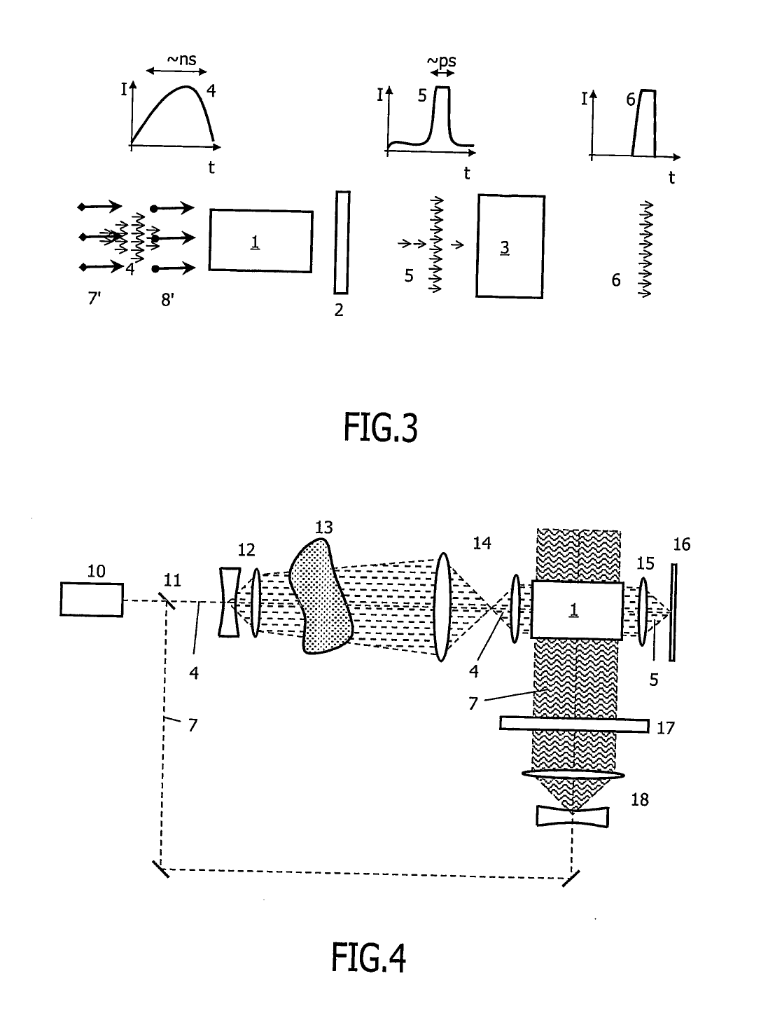

FIG. 3 shows a variant of the method of FIG. 2, in which the pump pulse and the quench pulse are irradiated in parallel with the signal photons.

FIG. 4 shows a diagram of the apparatus used to image a light source hidden by a body.

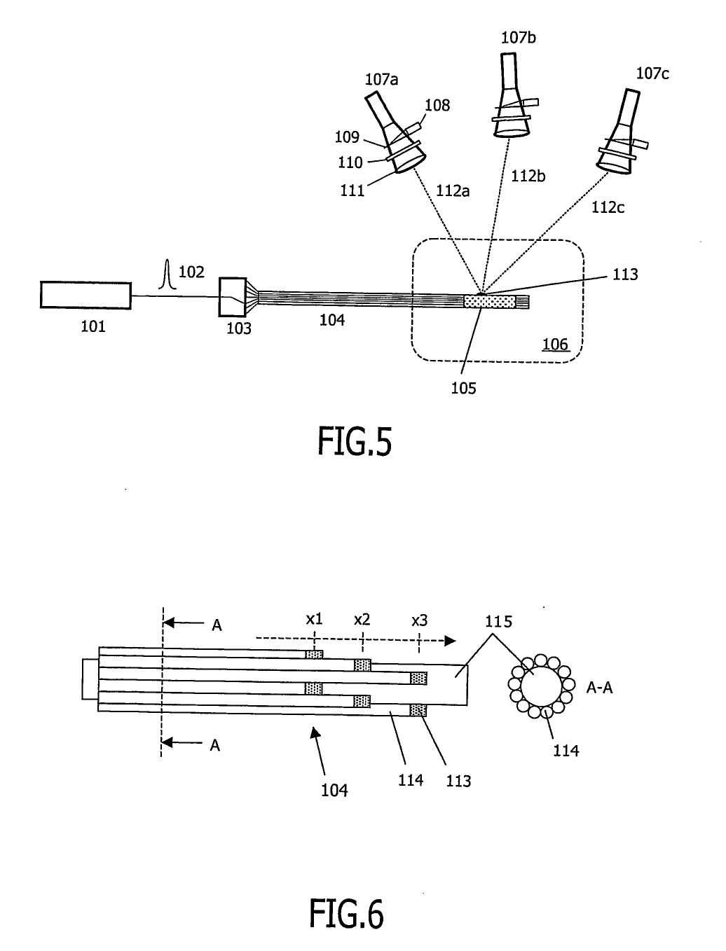

FIG. 5 schematically shows a set-up for locating a catheter inserted into the body.

FIG. 6 shows a side view and a cross section of a catheter suitable for the locating method.

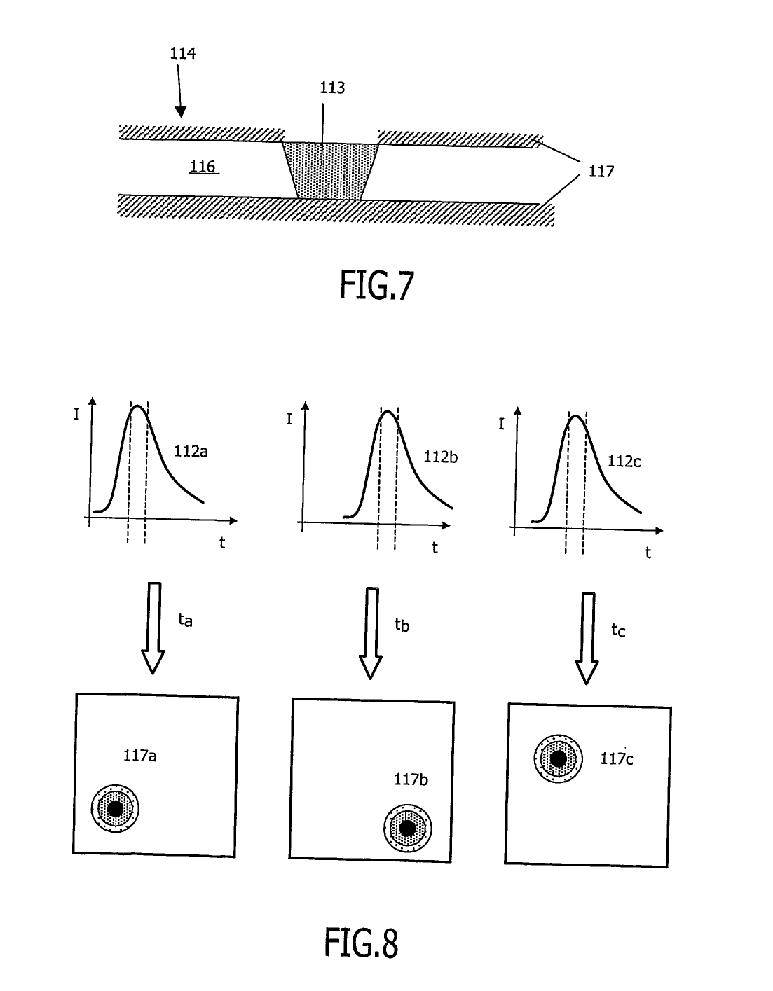

FIG. 7 shows a longitudinal section through a light guide of the catheter of FIG. 6.

FIG. 8 shows the imaging of NIR signal pulses on the detectors used.

FIG. 1 schematically shows the mode of operation of a novel method for the selective amplification of signal photons. The most important part of the associated set-up is an amplification medium 1, which may for example be a laser medium. The atoms or molecules of the amplification medium 1 may be converted to an excited state by irradiating pumping light of suitable pump frequency, as a result of which the population states of the medium with respect to the thermal equipartition are inverted. This procedure is referred to hereinbelow as “activation of the amplification medium”.

When signal photons 4 of suitable frequency are irradiated in, this results in an induced emission in the activated amplification medium 1, which induced emission leads to the desired amplification of the irradiated pulse of signal photons 4. The magnitude and the amplification of the medium 1 must in this case be selected in a suitable manner in order to allow good amplification of the signal (preferably in a single pass of the signal photons 4 through the amplification medium, although a number of passes are also possible) and hence allow use thereof in an imaging method. In this respect, for example, a laser medium 1 such as titanium:sapphire having a diameter of about 5 mm and a length (measured in the direction of the irradiated signal photons 4) of 20 mm is suitable. On account of the typically low intensity of the signal pulse 4, an exponential amplification response by the stimulated emission can be expected.

In the set-up shown in FIG. 1, a quench pulse 7 is passed through the amplification medium 1 perpendicular to the direction of incidence of the signal photons 4. The photons 7 of the quench pulse, by breaking down the excited states, bring about deactivation of the amplification medium 1. A high-power laser (e.g. Ti:Sa laser, not shown) having a pulse width of less than 1 ps may be used to generate the quench pulse 7. The intensity of such a laser is high enough to completely deactivate the amplification medium 1. The deactivation leads to the irradiated signal photons 4 no longer being amplified when they pass through the amplification medium 1 after the quench pulse 7. In this way, the quench pulse can be used to define the point in time until which amplification takes place in the amplification medium 1. The quench pulse 7 is preferably irradiated with a front that is inclined relative to the propagation direction of the signal photons 4, in order that the amplification of the signal photons 4 is “cut off” as precisely as possible with respect to the width of the amplification medium 1.

The signal pulse 4 is stretched by scattering processes generally to a duration of a number of nanoseconds in accordance with a geometric length of the pulse in the order of magnitude of 30 cm. A complete cross section of the amplification medium 1 perpendicular to the propagation direction of the signal pulse 4 is deactivated by the quench pulse 7 at a diameter of the amplification medium of 5 mm within 15 ps. By contrast, on account of capacitances, electrical resistances and geometric properties conventional photomultiplier tubes are limited to switching times of a number of nanoseconds. Compared to this, the proposed method represents an improvement of more than two orders of magnitude.

A bandpass filter 2 is arranged on the emergence side of the amplification medium 1, by means of which bandpass filter principally a broadband signal of amplified spontaneous emission is suppressed which is not in any temporal correlation with respect to the signal pulse 4 and is emitted spontaneously by the amplification medium 1 as long as the latter is in an activated state. The amplified signal pulse 5 leaving the spectral filter 2 has the profile shown schematically in the associated central diagram (intensity I over time t), in which the leading edge of the original signal pulse 4 is amplified compared to the rest of the signal, with a width in the picosecond range. In order to emphasize this intensity peak even more, the signal pulse 5 is passed through a saturable absorber 3, which only allows through the photons which lie above its saturation limit. The saturable absorber 3 may be for example a saturable absorber mirror of semiconductor material (SESAM) (cf. Keller, U., Miller, D. A. B., Boyd, G. D., Chiu, T. H., Ferguson, I. F., Asom, M. T., Opt. Lett. 17, 505 (1992); U. Keller, K. J. Weingarten, F. X. Kärtner, D. Kopf, B. Braun, I. D. Jung, R. Fluck, C. Hönninger, N. Matuschek, J. Aus der Au, IEEE J. Sel. Top. Quantum Electron. 2, 435, (1996); U. Keller in Nonlinear Optics in Semiconductors, edited by E. Garmire and A. Kost (Academic, Boston, Mass., 1999), Vol. 58, p. 211). Depending on the amplification factor, other intensity filters could also be used. If the amplification is very high, the step of intensity filtering may where appropriate also be completely omitted.

FIG. 2 shows a further developed set-up for carrying out a selective amplification. The essential difference with respect to the set-up of FIG. 1 is that the amplification medium 1 is activated by a pump pulse 8 of light of suitable pump frequency. In the example shown, the pump pulse 8, like the quench pulse, is irradiated perpendicular to the propagation direction of the signal photons 4 that are to be amplified. The amplification medium 1, which is initially inactive, is activated by the pump pulse 8 at a desired point in time with light velocity, as a result of which the start of the time window for the amplification can be defined. In particular, the amplification can in this way take place in a central region of the signal pulse 4. The remainder of the method comprising the spectral filtering by the filter 2 and the absorption of unamplified signal photons by the saturable absorber 3 is analogous to FIG. 1.

FIG. 3 shows a further variant. The difference with respect to the set-ups of FIG. 1 and FIG. 2 is that the pump pulse 8′ (if such a pulse is used) and the quench pulse 7′ are irradiated into the amplification medium 1 approximately parallel to the signal pulse 4. There should be a slight inclination of typically about 0° to about 20° between the propagation directions of pump pulse and quench pulse on the one hand and signal pulse on the other hand, in order to avoid an undesirable mixing of the rays on the output side. Furthermore, the pump pulse 8′ and the quench pulse 7′ are preferably broadband whereas the signal is narrow-band, in order that the separation of signal pulse on the one hand and quench pulse/pump pulse on the other hand, by means of a spectral filter, is more easily possible.

By means of the approximately parallel running of the wave fronts of signal pulse 4, pump pulse 8′ and quench pulse 7′, it is possible, from the signal pulse, for photons having a high selectivity from a desired time window to be amplified selectively. In the case of approximately planar waves, signal photons of the same time window all lie for example in the same plane or planar layer, which can be located very precisely between two planar wave fronts of pump pulse and quench pulse. The precise definition of the time window can moreover be used to select the width of the time window to be very small (typically in the order of magnitude of femtoseconds).

FIG. 4 schematically shows one specific use of the proposed method for the selective amplification of signal photons. The set-up shown comprises as light source a laser 10 which transmits short light pulses having a duration in the order of magnitude of nanoseconds and a frequency in the near infrared NIR range (0.65 μm to 3 μm). The light pulse of the laser 10 is split by a beam splitter 11 into a signal pulse 4 and a quench pulse 7 (alternatively the quench pulse 7 could also be generated by a separate laser). The signal pulse 4 is passed over suitable optics 12 onto or through an object 13 that is to be examined, such as a tissue sample for example, and then shaped by further optics 14 to form a parallel bundle of rays, said bundle of rays passing in the longitudinal direction through an amplification medium 1 of the type described in FIGS. 1 to 3. The (amplified) emission light 5 leaving the amplification medium 1 is bundled by further optics 15 on a detector plane 16, for example a CCD chip, to generate a geometric image.

The quench pulse 7 generated at the beam splitter 11 is passed via tilted mirrors and optics 18 such that it passes through the amplification medium 1 as a parallel bundle of rays perpendicular to the direction of the signal bundle 4. A phase shifter 17 may additionally be placed between the optics 18 and the amplification medium 1. The point in time at which the quench pulse 7 passes through the amplification medium 1 relative to the signal pulse 4 can be set by the length of the light path of the quench pulse 7 from the beam splitter 11 to the amplification medium 1. The amplification medium 1 is thus operated as a selective time window filter unit in the manner described in general terms in FIGS. 1 to 3. That is to say that the activated amplification medium 1 amplifies the irradiated signal pulse 4 until said amplification medium is deactivated following the arrival of the quench pulse 7.

FIG. 4 shows, not in detail, a filter (e.g. spectral bandpass filter, polarization filter, intensity filter or a combination thereof) and a saturable absorber between the optics 15 and the detector 16. By means of the spectral filter, spontaneous emissions of the amplification medium 1 can be screened out. The saturable absorber serves to screen out unamplified fractions of the signal pulse 4.

The optical imaging of a biological tissue 13 for example by means of NIR light is very difficult on account of the high scattering rates in these media. Photons having optical wavelengths are scattered to such a great extent that the probability of multiple scattering is also very high. Imaging with a high spatial resolution therefore requires means for screening out scattered signal photons. On account of the high fraction of multiple-scattered signal photons, collimators directed at the signal source (as in X-ray computer-aided tomography) cannot be used in this respect since on account of the multiple scattering scattered photons may again come from the direction of the signal source. On the other hand—also in view of the availability of coherent, monochromatic high-power lasers at optical wavelengths—the use of optical measurement methods is desirable in medicine since the signal photons at optical wavelengths that are used are not harmful to biological tissue, unlike X-ray radiation for example. Against this background, the abovementioned method offers an advantageous solution since it permits the screening-out of scattered photons by defining a suitable time window.

Besides scattering, the absorption of optical signal photons in biological tissue is also a source of interference. However, this interference can be compensated by using suitable wavelengths such as NIR for example or by relatively long recording durations, which are readily possible on account of the fact that the radiation is not harmful.

Rather than for the generation of a two-dimensional image in the detector plane 16, the set-up shown in FIG. 4 can also be used for (“zero-dimensional”) absorption measurements. Such measurements may also be carried out on a number of lines. Furthermore, the method can be expanded to a tomographic image generation system (cf. Schmidt, F. E. W., Development of a Time-Resolved Optical Tomography System for Neonatal Brain Imaging, PhD thesis, University College London, 1999; Huijuan Zhaol, Feng Gaol, Yukari Tanikawa, Yoichi Onodera, Masato Ohmi, Masamitsu Haruna and Yukio Yamada, Imaging of in vitro chicken leg using time-resolved near-infrared optical tomography, Phys. Med. Biol. 47 (2002) 1979-1993) or be used in the field of “optical computing” or as a pulse picker.

The present invention thus provides a technology which permits the precise amplification of very short section of light pulses. This may be used to aid imaging methods which are based on a differencing of the propagation time of signal photons having a high temporal and spatial resolution. The method is particularly suitable for the optical imaging of highly heterogeneous media, in which there is a high degree of scattering of signal photons having optical wavelengths.

A fundamental principle of the invention is the use of an active amplification medium to amplify the signal pulse, where short laser pulses switch the amplification of the medium on and off as the signal pulse passes through, in order to amplify only a very short time slice of the signal pulse. This switching is made possible by a rapid pumping and/or quenching of an amplification medium with a reference laser pulse. In order to amplify the leading edge of a signal pulse, only one quench pulse is required which may be generated by the same laser as the signal pulse or by a separate laser.

The locating of a catheter will be described in more detail below with reference to FIGS. 5 to 8. In this respect, FIG. 5 schematically shows a catheter 104 which has been inserted into a volume of interest 106, such as the heart region of a patient for example. In order to be able to monitor the use of the catheter 104 and the carrying out of diagnostic and/or therapeutic measures, it is important to locate the catheter or at least a relevant section thereof (e.g. the tip) as precisely as possible. One such location operation is achieved according to the invention by the emission of NIR light from an emission section 105 of the catheter 104 and detection thereof outside the body. The detection is carried out by a number of cameras 107a, 107b, 107c from which images can be taken with the aid of stereoscopic methods as to the location of the emitting section 105. One embodiment of this principle that is shown in the figures is described in more detail below.

The tip of the catheter 104 that is to be located by means of the method is shown schematically in FIG. 6 in a side view (on the left) and in cross section along the line A-A (on the right). The catheter 104 has a number of typically 100 NIR light guides 114 which are arranged around the catheter core 115. For reasons of clarity, only much fewer light guides are shown in FIG. 6. The core 115 of the catheter 104 is of no independent significance for the locating method currently under consideration. It may be used to accommodate other catheter functions, a guidewire or the like.

The light guides 114 are modified in that at the end they have short sections 113 that have a length of about 100 μm and contain or are composed of a material that scatters NIR radiation to a great extent. FIG. 7 shows such a scattering section 113 in a longitudinal section through a light guide 114. The scattering section 113 should be dense enough to ensure an isotropic emission of the NIR radiation and hence virtually constant signal strengths for all orientations of the catheter, and also prevent measurement errors. The scattering sections are preferably formed by moving the sheath 117 and the core 116 of a light guide 114 away from one another over a length of about 100 μm, with the resulting gap then being filled with an NIR-scattering material. A scattering efficiency of 100% is to be desired in this case. A suitable material is for example an adhesive including small particles or gas bubbles, as a result of which very dense variations of the refractive index are generated.

With 100 light guides 114, for example ten different axial positions xᵢ (FIG. 6) could be produced on a catheter of 3 French (i.e. about 1 mm diameter), with ten emission points 113 distributed in a ring-like manner over the circumference being involved at each axial position. As an alternative, 100 emission points distributed in a ring-like manner over the circumference could be formed at a single axial position, in order to be able to trace for example in a targeted manner a specific point such as the catheter tip for example. The diameter of the light guide would in this case typically be 50 μm, and this corresponds to the size of commercially available light guides.

As can be seen in FIG. 5, the locating device comprises a laser 101 which provides NIR laser pulses 102 having a wavelength of typically 800 nm and a pulse duration of about 1 ps or less (corresponding to a pulse length of 300 μm). These light pulses 102 are passed to a light guide switch 103 and from there optionally fed into an individual light guide (or into a group of light guides) of the catheter 104. The light guide switch 103 permits switching rates in the kHz to MHz range. By actuating this switch 103, it is possible for light pulses 102 coming sequentially from the laser 101 to be transmitted into the various light guides 114 of the catheter 104. From there they are transported to the tip 105 of the catheter, the position of which tip is to be located. Upon reaching the scattering sections 113 on the catheter tip, the laser pulses 102 are isotropically emitted into the interior of the body volume 106.

Outside the body, (at least) three CCD cameras 107a, 107b and 107c are placed at various positions. The NIR light 112a, 112b, 112c transmitted from one emission point 113 to these cameras is picked up by the imaging optics of the cameras. Each of the optics comprises a spectral bandpass filter 110 for NIR light, an imaging element (e.g. a lens 111 or a concave mirror) and a beam splitter 109 (for example a mirror for NIR having a reflectivity of less than 100%, preferably 50%). The cameras 107a, 107b, 107c are in each case coupled to a suitable item of image processing hardware and/or software.

The detectors furthermore comprise image amplifiers and a time window filter unit (not shown) which may operate for example in accordance with the principle shown in FIGS. 1 to 4 and which makes it possible to take into account in a targeted manner only photons from a predefined time window. In particular, it is possible in this way to exclude from the detection photons which have been scattered in the body volume 106, since they arrive with a time delay with respect to the start of the received signal. The photons which arrive “on time” using the direct route are by contrast taken into account in the cameras 107a, 107b and 107c and combined to form a two-dimensional image of the emission point on the catheter 104. From the images thus generated in two or more cameras it is possible to determine the directions of incidence 112a, 112b, 112c of the direct radiation, from which in turn it is possible to locate the spatial position of the emission point 113 on the catheter 104.

The time window that is to be taken into account is determined for each camera 107a, 107b, 107c from a first light pulse with the aid of rapid photomultiplier tubes (PMTs) 108 which are provided at each camera. As can be seen in the schematic drawing of FIG. 8, the propagation times tₐ, t_{b} and t_{c} required by a photon emitted from the emission point 113 to reach the respective camera can be determined from the temporally offset profiles of the measured pulses. The next light pulse emitted by the laser 101 is then picked up by the cameras 107a, 107b, 107c, and this gives the desired two-dimensional images 117a, 117b, 117c of the emission point 113 in the image planes of the cameras. The images 117a, 117b, 117c generated by the detected photons are generally relatively undefined. However, this does not adversely affect the desired location operation, as long as the center point of the respective images can be determined with sufficient accuracy.

In a next step of the catheter location operation, the light guide switch 103 selects a different group of light guides 114 of the catheter 104, the emission points of which lie at a different axial position of the catheter 104, and the described method is repeated. This takes place until all the light guides of the catheter 104 have been processed.

The calculated positions of the emission points 113 of the catheter 104 can be compared with knowledge about the deformation properties of the catheter and/or about the shape of the organ in which the catheter is located. In this way, errors are reduced.

The locating of the catheter 104 is significantly influenced by the photon statistics, an estimate of which is given below. The following initial data are used as a basis: a bundle comprising 100 light guides; a desired refresh rate of the position information of 20 Hz for 10 points distributed over the catheter (that is to say 100/10=10 emission points per point to be located); a 1.5 W Ti:Sa laser; a collimator opening for each camera of about 10⁻⁴ steradian; CCD cameras 107a, 107b, 107c having a quantum efficiency of 20%; an overall light guide transparency of 10%; and a time window for the light pulse in the picosecond range, where the light pulse is stretched in the medium to about one nanometer by means of scattering processes. In this case, about 10⁸ photons may be expected for each camera, each point to be located and each image. These photons arrive at a CCD chip in the order of magnitude of, for example, 500×500 pixels. Each of the three cameras 107a, 107b, 107c detects a complete projection of the volume of interest 106, which has a size of typically 200×200×200 mm³ for example in the case of cardiac examinations. The lateral position of the signal of a camera therefore reflects the projected two-dimensional position of the emission point for the corresponding viewing angle. According to the strength of the photon signal specified above, the spatial resolution of the two-dimensional position determination for an ideal (i.e. punctiform) emission point, which by virtue of scattering and defocusing leads to a blurred signal distribution in each camera, is as expected very high (<100 μm). The focusing depth of each camera and of the optics is in this case adapted to the dimension of the volume of interest. A penetration depth of up to 500 mm may be expected, depending of the type of tissue passed through.

In some applications, a modulation of the refractive index may possibly be carried out if an improvement in the image quality by suppressing scattering processes is necessary (cf. V. V. Tuchin, I. L. Maksimova, D. A. Zimnyakov, I. L. Kon, A. H. Mavlutov, A. A. Mishin, “Light propagation in tissues with controlled optical properties”, J. of Biomedical Optics 1997, 2(4), pp. 401-417).

As an alternative to the set-up comprising three cameras 107a, 107b, 107c shown in FIG. 5, it is also possible to use just two 2D CCD cameras or three 1D CCD arrangements having cylindrical lenses.

The size of the volume 106 that can be examined is limited by the imaging device or the optical arrangement. However, the position of this volume 106 can be varied at will by moving the entire detector assembly. In this respect, self-adaptation is possible in particular, by comparing the number of points to be traced with the number of signals received and the reconstructed path of these signals. From this information it is possible to estimate the necessary movement (magnitude and direction) of the imaging device.

The set-up according to the invention can be expanded in a simple manner to a combined technology which allows robust and precise location and photodynamic therapy measures in the same device. For this purpose, the core 115 of the catheter 104 may comprise additional light guides which transport the light (UV light) necessary for photodynamic therapy.

1. A method of locating an instrument within a body comprising:
a) emitting NIR radiation from at least one emission point of the instrument;
b) detecting the emitted NIR radiation outside the body; and
c) reconstructing the position of the emission point from the detected NIR radiation.

2. The method as claimed in claim 1, wherein the detection of the emitted NIR radiation takes place at a number of locations outside the body and the position of the emission point is reconstructed stereoscopically.

3. The method as claimed in claim 1, wherein the NIR radiation is emitted sequentially by various emission points of the instrument.

4. The method as claimed in claim 1, wherein the NIR radiation is emitted as a short time pulse shaving a duration of between approximately 0.1 and approximately 10 ps.

5. The method as claimed in claim 1, wherein only photons of direct radiation are used to detect the NIR radiation outside the body.

6. The method as claimed in claim 1, wherein the photons of the emitted NIR radiation are passed into an activated amplification medium into which a deactivating quench pulse is irradiated in order to terminate the amplification.

7. A device for locating an instrument within a body comprising:
a) at least one detector for the detection of NIR radiation coming from at least one emission point of the instrument;
b) means for reconstructing the position of the emission point from the measured values of the detector.

8. The device as claimed in claim 7, wherein the detector has a time window filter unit for the selective detection of photons from a predefined time window.

9. The device as claimed in claim 8, wherein the time window filter unit is formed by an activatable amplification medium and a quenching device for irradiating a quench pulse into the amplification medium.

10. A catheter for use in a method as claimed in claim 1, comprising a number of NIR light guides which each have at least one NIR light-scattering section that acts as emission point and an inlet for the coupling-in of NIR pulses.

11. A device for locating an instrument within a body comprising:
(a) a plurality of light guides attached to the instrument, each light guide including an emission point;
(b) a light pulse source that produces NIR light pulses that selectively pass through a set of the plurality of light guides until the NIR light pulses reach an emission point, wherein the NIR light pulses are emitted into the body; and
(c) one or more detectors for detecting the NIR light pulses outside the body.

12. The device of claim 11 further comprising means for reconstructing the position of the emission point from information received by the one or more detectors.

13. The device of claim 11, wherein the one or more detectors includes a filter for selective detection of photons within a predefined time window.

14. The device of claim 13, wherein the filter comprises an activatable amplification medium and a quenching device for irradiating a quench pulse into the amplification medium.

15. The device of claim 14 further comprising a saturable absorber.

16. The device of claim 11, wherein the instrument is a catheter.

17. The device of claim 11, wherein the plurality of light guides are circumferentially positioned around the instrument.

18. The device of claim 17, wherein the emitting points of the plurality of light guides are axially staggered around the instrument.

19. The device of claim 11 further comprising a switching circuit, wherein said switching circuit determines which of the plurality of light guides the NIR light pulses will pass through.
